SEQUENCE LISTING (8 sequences)

SEQ ID NO: 1 - DNA, 40 bp, Artificial Sequence
  Synthetic Construct
tacgcgtcgacgcttcttttatgtagtattatttcagaag

SEQ ID NO: 2 - DNA, 36 bp, Artificial Sequence
  Synthetic Construct
tacgcgtcgacgccagtaaaagagattgtttctagc

SEQ ID NO: 3 - DNA, 2589 bp, Artificial Sequence
  Synthetic Construct
atgaaagtcacaacagtaaaggaattagatgaaaaactcaaggtaattaaagaagctcaaaaaaaattctcttgttactcgcaagaaatggttgatgaaatctttagaaatgcagcaatggcagcaatcgacgcaaggatagagctagcaaaagcagctgttttggaaaccggtatgggcttagttgaagacaaggttataaaaaatcattttgcaggcgaatacatctataacaaatataaggatgaaaaaacctgcggtataattgaacgaaatgaaccctacggaattacaaaaatagcagaacctataggagttgtagctgctataatccctgtaacaaaccccacatcaacaacaatatttaaatccttaatatcccttaaaactagaaatggaattttcttttcgcctcacccaagggcaaaaaaatccacaatactagcagctaaaacaatacttgatgcagccgttaagagtggtgccccggaaaatataataggttggatagatgaaccttcaattgaactaactcaatatttaatgcaaaaagcagatataacccttgcaactggtggtccctcactagttaaatctgcttattcttccggaaaaccagcaataggtgttggtccgggtaacaccccagtaataattgatgaatctgctcatataaaaatggcagtaagttcaattatattatccaaaacctatgataatggtgttatatgtgcttctgaacaatctgtaatagtcttaaaatccatatataacaaggtaaaagatgagttccaagaaagaggagcttatataataaagaaaaacgaattggataaagtccgtgaagtgatttttaaagatggatccgtaaaccctaaaatagtcggacagtcagcttatactatagcagctatggctggcataaaagtacctaaaaccacaagaatattaataggagaagttacctccttaggtgaagaagaaccttttgcccacgaaaaactatctcctgttttggctatgtatgaggctgacaattttgatgatgctttaaaaaaagcagtaactctaataaacttaggaggcctcggccatacctcaggaatatatgcagatgaaataaaagcacgagataaaatagatagatttagtagtgccatgaaaaccgtaagaacctttgtaaatatcccaacctcacaaggtgcaagtggagatctatataattttagaataccaccttctttcacgcttggctgcggattttggggaggaaattctgtttccgagaatgttggtccaaaacatcttttgaatattaaaaccgtagctgaaaggagagaaaacatgctttggtttagagttccacataaagtatattttaagttcggttgtcttcaatttgctttaaaagatttaaaagatctaaagaaaaaaagagcctttatagttactgatagtgacccctataatttaaactatgttgattcaataataaaaatacttgagcacctagatattgattttaaagtatttaataaggttggaagagaagctgatcttaaaaccataaaaaaagcaactgaagaaatgtcctcctttatgccagacactataatagctttaggtggtacccctgaaatgagctctgcaaagctaatgtgggtactatatgaacatccagaagtaaaatttgaagatcttgcaataaaatttatggacataagaaagagaatatatactttcccaaaactcggtaaaaaggctatgttagttgcaattacaacttctgctggttccggttctgaggttactccttttgctttagtaactgacaataacactggaaataagtacatgttagcagattatgaaatgacaccaaatatggcaattgtagatgcagaacttatgatgaaaatgccaaagggattaaccgcttattcaggtatagatgcactagtaaatagtatagaagcatacacatccgtatatgcttcagaatacacaaacggactagcactagaggcaatacgattaatatttaaatatttgcctgaggcttacaaaaacggaagaaccaatgaaaaagcaagagagaaaatggctcacgcttcaactatggcaggtatggcatccgctaatgcatttctaggtctatgtcattccatggcaataaaattaagttcagaacacaatattcctagtggcattgccaatgcattactaatagaagaagtaataaaatttaacgcagttgataatcctgtaaaacaagccccttgcccacaatataagtatccaaacaccatatttagatatgctcgaattgcagattatataaagcttggaggaaatactgatgaggaaaaggtagatctcttaattaacaaaatacatgaactaaaaaaagctttaaatataccaacttcaataaaggatgcaggtgttttggaggaaaacttctattcctcccttgatagaatatctgaacttgcactagatgatcaatgcacaggcgctaatcctagatttcctcttacaagtgagataaaagaaatgtatataaattgttttaaaaaacaaccttaa

SEQ ID NO: 4 - DNA, 657 bp, Artificial Sequence
  Synthetic Construct
atgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataa

SEQ ID NO: 5 - DNA, 666 bp, Artificial Sequence
  Synthetic Construct
atgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttag

SEQ ID NO: 6 - protein, 862 aa, Artificial Sequence
  Synthetic Construct
MKVTTVKELDEKLKVIKEAQKKFSCYSQEMVDEIFRNAAMAAIDARIELAKAAVLETGMGLVEDKVIKNHFAGEYIYNKYKDEKTCGIIERNEPYGITKIAEPIGVVAAIIPVTNPTSTTIFKSLISLKTRNGIFFSPHPRAKKSTILAAKTILDAAVKSGAPENIIGWIDEPSIELTQYLMQKADITLATGGPSLVKSAYSSGKPAIGVGPGNTPVIIDESAHIKMAVSSIILSKTYDNGVICASEQSVIVLKSIYNKVKDEFQERGAYIIKKNELDKVREVIFKDGSVNPKIVGQSAYTIAAMAGIKVPKTTRILIGEVTSLGEEEPFAHEKLSPVLAMYEADNFDDALKKAVTLINLGGLGHTSGIYADEIKARDKIDRFSSAMKTVRTFVNIPTSQGASGDLYNFRIPPSFTLGCGFWGGNSVSENVGPKHLLNIKTVAERRENMLWFRVPHKVYFKFGCLQFALKDLKDLKKKRAFIVTDSDPYNLNYVDSIIKILEHLDIDFKVFNKVGREADLKTIKKATEEMSSFMPDTIIALGGTPEMSSAKLMWVLYEHPEVKFEDLAIKFMDIRKRIYTFPKLGKKAMLVAITTSAGSGSEVTPFALVTDNNTGNKYMLADYEMTPNMAIVDAELMMKMPKGLTAYSGIDALVNSIEAYTSVYASEYTNGLALEAIRLIFKYLPEAYKNGRTNEKAREKMAHASTMAGMASANAFLGLCHSMAIKLSSEHNIPSGIANALLIEEVIKFNAVDNPVKQAPCPQYKYPNTIFRYARIADYIKLGGNTDEEKVDLLINKIHELKKALNIPTSIKDAGVLEENFYSSLDRISELALDDQCTGANPRFPLTSEIKEMYINCFKKQP

SEQ ID NO: 7 - protein, 218 aa, Artificial Sequence
  Synthetic Construct
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 8 - protein, 221 aa, Artificial Sequence
  Synthetic Construct
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

(19) United States
(12) Patent Application Publication
(10) Pub. No.: US 2011/0027845 A1
(43) Pub. Date: Feb. 3, 2011

(54) ENHANCED ETHANOL AND BUTANOL PRODUCING MICROORGANISMS AND METHOD FOR PREPARING ETHANOL AND BUTANOL USING THE SAME

(75) Inventors: Sang Yup Lee, Daejeon (KR); Yu-Sin Jang, Daejeon (KR); Jin Young Lee, Incheon (KR); Kwang Seop Jung, Daejeon (KR); Jae Hyun Kim, Daejeon (KR)

Correspondence Address: MOORE & VAN ALLEN PLLC, P.O. BOX 13706, Research Triangle Park, NC, 27709, US

(73) Assignees: KOREA ADVANCED INSTITUTE OF SCIENCE AND TECHNOLOGY, Daejeon (KR); BIOFUELCHEM CO., Daejeon (KR); GS CALTEX CORPORATION, Seoul (KR)

(21) Appl. No.: 12/809,323

(22) PCT Filed: Dec. 22, 2008
(86) PCT No.: PCT/KR2008/007577

§ 371 Date: Aug. 13, 2010

(30) Foreign Application Priority Data
Dec. 20, 2007 (KR) 10-2007-0134701

(51) Int. Cl. C12P 7/16; C12N 1/00; C12N 1/21; C12P 7/06

(52) U.S. Cl. 435/160; 435/243; 435/252.3; 435/161

(57) ABSTRACT

The present invention relates to a recombinant microorganism having an enhanced ability to produce ethanol and butanol and a method for preparing ethanol and butanol using the same, and more particularly to a recombinant microorganism having an enhanced ability to produce ethanol and butanol, into which a gene encoding CoA transferase and a gene encoding alcohol/aldehyde dehydrogenase are introduced, and to a method for preparing ethanol and butanol using the same. The recombinant microorganism according to the present invention, obtained by manipulating metabolic pathways of microorganisms, is capable of producing butanol and ethanol exclusively without producing any byproduct, and thus is useful as a microorganism producing industrial solvents and transportation fuel.

### TECHNICAL FIELD

The present invention relates to a recombinant microorganism having an enhanced ability to produce ethanol and butanol and a method for preparing ethanol and butanol using the same, and more particularly to a recombinant microorganism having an enhanced ability to produce ethanol and butanol, into which a gene encoding CoA transferase and a gene encoding alcohol/aldehyde dehydrogenase are introduced, and a method for preparing ethanol and butanol using the same.

### BACKGROUND ART

Currently, ethanol and butanol has a huge market as industrial solvents, and the possibility of using them as fuel for the means of transportation such as automobiles and the like, are being realized, and thus, continuous increase in the demand for ethanol and butanol, is being expected.

Traditionally, ethanol (C₂H₅OH) has been prepared by a method of fermenting starch or sugars, and most alcoholic beverages theses days are prepared by such a method. However, except for the preparation of alcoholic beverages, ethanol is currently being prepared by synthetic methods comprising using ethylene (ethene) obtained from petroleum as a raw material: a sulfuric acid hydrolysis method in which ethylene is absorbed into sulfuric acid to produce the sulfuric acid ester of ethanol, then hydrolyzed to produce ethanol together with diethyl ether, and a direct hydration method in which ethylene in a gaseous phase is allowed to react with aqueous vapor by contact using a solid phosphoric acid catalyst, thereby leading to direct synthesis of ethanol. However, said methods have disadvantages in that petroleum is a basic raw material, and that in the case of the sulfuric acid hydrolysis method, large scale facilities are required for the concentration and circulation of a large amount of sulfuric acid.

Meanwhile, the worldwide production of butanol (C₄H₉OH) is estimated to be about 1.1 million tons/year. All the commercially available butanol today is produced by chemical synthesis. As in the case of ethanol, chemical synthesis of butanol also uses petroleum as a raw material to produce propylene, which is used to synthesize butanol by the oxo process. Such a method involving high temperature and high pressure, using petroleum as a raw material, is inefficient in both cost and energy (Tsuchida et al., Ind. Eng. Chem. Res., 45:8634, 2006). That is, the production of ethanol and butanol by means of petroleum chemistry has the problem of discharging large amounts of hazardous wastes, waste solutions and waste gases (including carbon monoxide) during the production process, and especially has a limitation that fossil fuel is used as a basic material.

As described above, most of the butanol produced so far has been produced by chemical synthesis. Although there has been a rapid increase of worldwide interests in the bio-ethanol and bio-butanol researches due to the rise of oil prices and accompanying environmental problems, there has been no example of efficiently producing bio-ethanol and bio-butanol exclusively yet.

So far, most of the methods for producing butanol and ethanol by fermentation have used Clostridium; and in one case, a plasmid (pFNK6) was prepared by introducing 3 genes: a gene (adc) encoding acetoacetic acid decarboxylase, a gene (ctfA) encoding CoA transferase A and a gene (ctfB) encoding CoA transferase B into a vector and constructing an artificial operon using an adc promoter, and the plasmid was introduced into Clostridium acetobutylicum ATCC 824, thereby improving the productivity of acetone, butanol and ethanol by 95%, 37% and 90%, respectively, compared to the wild-type (Mermelstein et al., Biotechnol. Bioeng., 42:1053, 1993). There is another case where cloning and overexpression of aad (alcohol/aldehyde dehydrogenase) resulted in relatively improved butanol and ethanol production compared to acetone production, compared to the wild-type (Nair et al., J. Bacteriol., 176:871, 1994). In addition, there has been an attempt that buk (butyrate kinase) and pta (phosphotransacetylase) were inactivated as a means of inactivating the functions of the genes, and it was reported that fermentation beyond pH 5.0 of a strain (PJC4BK), whose buk gene was inactivated, resulted in a remarkable increase in butanol production, up to 16.7 g/l (Harris et al., Biotechnol. Bioeng., 67:1, 2000). However, inactivation of pta was reported to have shown no significant difference in solvent production compared to the wild-type (Harris et al., Biotechnol. Bioeng., 67:1, 2000). Furthermore, Clostridium beijerinckii BA101, which is a mutant strain obtained through random mutagenesis, was fermented using maltodextrins as a carbon source, and was reported to have produced 18.6 g/l of butanol (Ezeji et al., Appl. Microbiol. Biotechnol., 63:653, 2004). However, the above results are examples of producing butanol and ethanol together with acetone as a byproduct, and has a disadvantage in that they can not be used as fuel without removing acetone, because of the properties of acetone.

There is a case of producing ethanol and butanol without acetone production using a recombinant microorganism, which was constructed by introducing aad (alcohol/aldehyde dehydrogenase) into a mutant strain of Clostridium acetobutylicum defective in the functions of all of adc (a gene encoding acetoacetic acid decarboxylase), ctfA (a gene encoding CoA transferase A), ctfB (a gene encoding CoA transferase B) and aad (a gene encoding alcohol/aldehyde dehydrogenase); however, the method has a problem of low productivity, since the final concentrations of butanol and ethanol were 84 mM and 8 mM, respectively (Nair et al., J. Bacteriol., 176:5843, 1994). There is also another case of producing butanol, by introducing a recombinant vector carrying genes of Clostridium acetobutylicum into a strain of E. coli (Shota et al., Metab. Eng., In Press, 2007) but the maximum concentration of the produced butanol was low with a concentration of 552 mg/l, making its industrial use impossible.

Therefore, there is an urgent need for the development of microorganisms which can produce butanol or a mixture of ethanol and butanol with high efficiency without producing byproducts, such as acetone, so that they can be directly used as fuel.

Accordingly, the present inventors have made extensive efforts to develop microorganisms capable of producing ethanol and butanol with high yield without producing byproducts based on the pathway for ethanol and butanol synthesis (FIG. 1), and as a result, constructed a recombinant microorganism by cloning two enzymes derived from Clostridium acetobutylicum ATCC 824: (1) ctfAB encoding CoA transferase, which converts acetic acid and butyric acid into acetyl CoA and butylyl CoA, respectively, and (2) adhE1 encoding alcohol/aldehyde dehydrogenase, which converts acetyl CoA and butyryl CoA into ethanol and butanol, respectively, and introducing the cloned genes into a host microorganism incapable of producing organic solvents, and confirmed that the recombinant microorganism produces high concentrations of ethanol and butanol while producing almost no acetone as a byproduct, thus completing the present invention.

### SUMMARY OF INVENTION

Therefore, it is a main object of the present invention to provide a recombinant microorganism producing butanol or ethanol/butanol with high efficiency without producing byproducts, and a method for constructing the same.

Another object of the present invention is to provide a method for preparing ethanol and butanol using said recombinant microorganism.

In order to achieve the above objects, the present invention provides a method for constructing a recombinant microorganism having an enhanced ability to produce ethanol and butanol, the method comprises introducing a gene encoding an enzyme that converts acetic acid and butyric acid to acetyl CoA and butylyl CoA, respectively; and/or a gene encoding an enzyme that converts acetyl CoA and butyryl CoA to ethanol and butanol, respectively, into a host microorganism which has genes encoding enzymes involved in the biosynthetic pathway for conversion of acetyl CoA to butyryl CoA.

The present invention also provides a recombinant microorganism having an enhanced ability to produce ethanol and butanol, which has a gene encoding an enzyme that converts acetic acid and butyric acid to acetyl CoA and butylyl CoA, respectively; and/or a gene encoding an enzyme that converts acetyl CoA and butyryl CoA to ethanol and butanol, respectively, introduced or amplified into a host microorganism having genes encoding enzymes involved in the biosynthetic pathway for conversion of acetyl CoA to butyryl CoA.

In addition, the present invention provides a method for preparing ethanol and/or butanol, the method comprising the steps of culturing said recombinant microorganism and recovering ethanol and/or butanol from the culture broth.

Other features and aspects of the present invention will be more apparent from the following detailed description and the appended claims.

### BRIEF DESCRIPTION OF DRAWINGS

FIG. 1 is a schematic diagram showing the metabolic pathway in a degenerated strain of Clostridium acetobutylicum (A), which has no ability to produce ethanol and butanol, and the metabolic pathway for the synthesis of ethanol and butanol in a recombinant strain constructed by introducing ctfAB and adhE1 into the degenerated strain (B).

FIG. 2 is a genetic map of the recombinant vector pIMP1::adhE1.ctfAB which contains ctfAB and adhE1.

### DETAILED DESCRIPTION OF THE INVENTION AND PREFERRED EMBODIMENTS

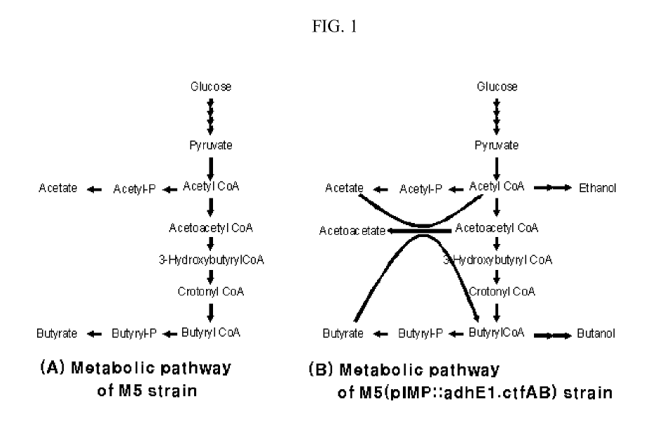

In the present invention, in order to develop a microorganism capable of producing ethanol/butanol with high yield without producing byproducts such as acetone, based on the pathway for ethanol and butanol synthesis (FIG. 1), the following two enzymes derived from Clostridium acetobutylicum ATCC 824 were cloned: (1) ctfAB encoding CoA transferase, which converts acetic acid and butyric acid into acetyl CoA and butylyl CoA, respectively, and (2) adhE1 encoding alcohol/aldehyde dehydrogenase, which converts acetyl CoA and butyryl CoA into ethanol and butanol, respectively, and then the cloned genes were introduced into a host microorganism which has genes encoding enzymes involved in the biosynthetic pathway for conversion of acetyl CoA to butyryl CoA and has no ability to produce organic solvents such as acetone, thus constructing a recombinant microorganism.

Therefore, the present invention relates to method for constructing a recombinant microorganism having an enhanced ability to produce ethanol and butanol, the method comprises introducing or amplifying a gene encoding an enzyme that converts acetic acid and butyric acid to acetyl CoA and butylyl CoA, respectively; and/or a gene encoding an enzyme that converts acetyl CoA and butyryl CoA to ethanol and butanol, respectively, into a host microorganism which has genes encoding enzymes involved in the biosynthetic pathway for conversion of acetyl CoA to butyryl CoA.

The present invention also relates to a recombinant microorganism having an enhanced ability to produce ethanol and butanol, which has a gene encoding an enzyme that converts acetic acid and butyric acid to acetyl CoA and butylyl CoA, respectively; and/or a gene encoding an enzyme that converts acetyl CoA and butyryl CoA to ethanol and butanol, respectively, introduced or amplified into a host microorganism having genes encoding enzymes involved in the biosynthetic pathway for conversion of acetyl CoA to butyryl CoA.

In the present invention, the term “amplification” is used herein broadly to refer to processes: mutation, substitution or deletion, and insertion of some base(s) of a relevant gene; or introducing a gene derived from other microorganism encoding the same enzyme to increase the activity of the corresponding enzyme.

In the present invention, said biosynthetic pathway for conversion of acetyl CoA into butyryl CoA is preferably [acetyl CoA→acetoacetyl CoA→3-hydroxybutyryl CoA→crotonyl CoA→butyryl CoA].

In the present invention, the host microorganism preferably has an acetone biosynthetic pathway blocked and thus has acetone production of less than 10% of the total organic solvent production. An adc (a gene encoding acetoacetic acid decarboxylase) may be deleted in said pathway for acetone biosynthesis, but is not limited thereto. And said host microorganism is preferably derived from the genus Clostridium, but it is not limited thereto as long as it has a biosynthetic pathway for conversion of acetyl CoA into butyryl CoA.

In the present invention, preferably the enzyme converting acetic acid and butyric acid into acetyl CoA and butylyl CoA, respectively, is CoA transferase; and the gene encoding the CoA transferase is ctfAB. Also, preferably the enzyme converting acetyl CoA and butyryl CoA into ethanol and butanol, respectively, is alcohol/aldehyde dehydrogenase; and the gene encoding the alcohol/aldehyde dehydrogenase is adhE1. The present invention used only said ctfAB and adhE1 derived from Clostridium acetobutylicum ATCC 824 as an example, but genes derived from other microorganisms may be used without limitation as long as they are expressed in a host cell, into which they are introduced, and have the same activities.

In the examples of the present invention, the host microorganism used is a mutant M5 strain of Clostridium acetobutylicum which lacks megaplasmid (carrying 127 genes, including a gene encoding acetoacetic acid decarboxylase, a gene encoding CoA transferase and a gene encoding alcohol/aldehyde dehydrogenase). The mutant M5 strain of Clostridium acetobutylicum is a microorganism whose pathway for acetone biosynthesis is blocked (FIG. 1). In the present invention, only Clostridium acetobutylicum M5 was used as an example of the host microorganisms of the genus Clostridium whose pathway for acetone biosynthesis is blocked, but Clostridium acetobutylicum 1NYG, 4NYG, 5NYG and DG1 (Stim-Herndon, K. P. et al., Biotechnol./Food Microbiol., 2:11, 1996), C. acetobutylicum ATCC 824 Type IV, M3, M5, 2-BB R, 2-BB D, Rif B12, Rif D10, Rif F7, and C. butyricum ATCC 860 (Clark, S. W. et al., Appl. Environ. Microbiol., 55:970, 1989) may also be used. In the present invention, it was confirmed that when the recombinant microorganism M5(pIMP1::adhE1.ctfAB) was constructed by introducing a recombinant vector (pIMP1::adhE1.ctfAB) carrying said ctfAB and adhE1 into said host microorganism, an cultured, it produces high concentrations of butanol/ethanol, while producing almost no acetone.

Therefore, in another aspect, the present invention relates to a method for preparing ethanol and/or butanol, the method comprising the steps of culturing said recombinant microorganism and recovering ethanol and/or butanol from the culture broth.

In the present invention, the processes of culturing recombinant microorganisms and recovering ethanol and butanol may be performed using the conventional culture method and the conventional method for isolation and purification of ethanol/butanol known in the fermentation art. In addition, although the recovery of butanol and ethanol is usually carried out after completing the culture, it may be carried out during culture in order to improve productivity, using proper methods such as gas-stripping method (Thaddeus et al., Bioprocess Biosyst. Eng., 27:207, 2005). That is, continuous culture while recovering ethanol and butanol produced during the culture is also within the scope of the present invention.

On the other hand, although the present invention illustrated only a case where a pathway for butanol biosynthesis was blocked, there is a report on the improvement of butanol production by blocking the pathway for butyrate biosynthesis in a strain of Clostridium acetobutylicum ATCC 824 (Harris et al., Biotechnol. Bioeng., 67:1, 2000); therefore it could be inferred that production of ethanol and butanol could be improved by blocking the biosynthetic pathway for conversion of butyryl CoA into butyrate in the metabolic pathway of FIG. 1. As an alternative method, introduction of genes capable of utilizing acetate, such as acs and atoDA, may also improve ethanol and butanol production.

### EXAMPLES

Hereinafter, the present invention will be described in further detail with reference to examples. It is to be understood, however, that these examples are for illustrative purposes only and are not to be construed to limit the scope of the present invention.

Especially, the following examples illustrate a specific mutant strain of Clostridium acetobutylicum M5 as a host strain incapable of producing organic solvents, but it will be apparent to one skilled in the art that other microorganisms of the genus Clostridium or of other genera, which have biosynthetic pathways for conversion of acetyl CoA to butyryl CoA and whose pathways for organic solvent biosynthesis are blocked can be used as a host strain, and the same genes can be introduced into the host strain for ethanol and butanol production.

### Example 1

### Preparation of a Recombinant Vector Containing adhE1 Gene Encoding Alcohol/Aldehyde Dehydrogenase, and ctfAB Gene Encoding CoA Transferase

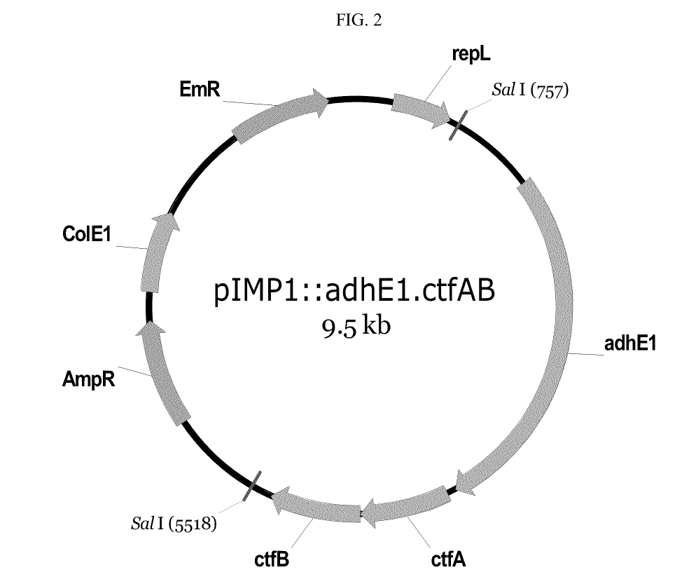

The adhE1, ctfA and ctfB genes of Clostridium acetobutylicum ATCC 824, which have the base sequences of SEQ ID NO: 3, SEQ ID NO: 4 and SEQ ID NO: 5, respectively, were cloned together with the promoter and transcription termination sequences thereof. First, using chromosomal DNA of Clostridium acetobutylicum ATCC 824 as a template, PCR (Table 1) was performed with the primers of SEQ ID NO: 1 and SEQ ID NO: 2, then the obtained adhE1, ctfA and ctfB genes were cut with the restriction enzyme SalI and inserted into Clostridium/E. coli shuttle vector pIMP1 (Mermelstein, L. D. et al., Bio/Technol., 10:190, 1992) cut with the same restriction enzyme, thus preparing a recombinant vector pIMP1::adhE1 ctfAB (FIG. 2). Genes (adhE1, ctfAB) derived from Clostridium acetobutylicum ATCC 824, which encode alcohol/aldehyde dehydrogenase and CoA transferase, were thus cloned.

TABLE 1
PCR conditions

| | | Restriction site | Reaction condition |
|---|---|---|---|
| Gene | Primer | in primer | (polymerase: Pfu-x) |
| adhE1 | P1(SEQ ID NO: 1) | Sal I | Cycle I: 95° C., 5 min |
| and | P2(SEQ ID NO: 2) | | Cycle II: (30 cycles) |
| ctfAB | | | 95° C., 40 sec |
| | | | 61° C., 30 sec |
| | | | 72° C., 2.5 min |
| | | | Cycle III: 72° C., 5 min |
| | | | Cycle IV: 4° C., forever |

The base sequences of the cloned adhE1 and ctfAB genes, derived from Clostridium acetobutylicum ATCC 824, were analyzed, and the amino acid sequences of alcohol/aldehyde dehydrogenase and CoA transferase were deduced. As the result, the DNA sequences (SEQ ID NO: 3, SEQ ID NO: 4 and SEQ ID NO: 5) and amino acid sequences (SEQ ID NO: 6, SEQ ID NO: 7 and SEQ ID NO: 8) of the adhE1 and ctfAB of Clostridium acetobutylicum ATCC 824 were identified.

### Example 2

### Construction of Recombinant Microorganisms

M5(pIMP1::adhE1.ctfAB) strain was constructed by introducing the recombinant vector pIMP1::adhE1 ctfAB constructed in Example 1 into Clostridium acetobutylicum M5 strain by electroporation. First, the recombinant vector of Example 1 was introduced into Escherichia coli TOP10, which contains the vector pAN1 expressing Bacillus subtilis Phage Φ3T I methyltransferase (Mermelstein et al., Appl. Environ. Microbiol., 59:1077, 1993) to induce methylation thereof, such that the vector becomes suitable for transformation into Clostridium. The methylated vector was isolated and purified from E. coli, and then introduced into a mutant strain of Clostridium acetobutylicum M5 (Cornillot et al., J. Bacteriol., 179:5442, 1997) which lacks megaplasmid (carrying 176 genes, including a gene encoding acetoacetic acid decarboxylase, a gene encoding CoA transferase and a gene encoding alcohol/aldehyde dehydrogenase), thus preparing a recombinant microorganism. In addition, pIMP1, which had been used as a cloning vector, was introduced into Clostridium acetobutylicum M5 strain, thus preparing M5(pIMP1) strain.

M5 competent cells were prepared for transformation as follows: First, M5 strain was inoculated into 10 ml of CGM (Table 2) and cultured to an OD of 0.6. The culture broth was inoculated into 60 ml of 2×YTG medium (Bacto tryptone 16 g, Yeast extract 10 g, NaCl 4 g and Glucose 5 g per 1 liter) to a concentration of 10% and the cells were cultured for 4-5 hours. The microorganism cells were washed twice with transformation buffer (EPB, 270 mM sucrose 15 ml, 686 mM NaH₂PO₄ 110 μl, pH 7.4) and then suspended in 2.4 ml of the same buffer. The thus prepared 600 μl of the M5 competent cells were mixed with 25 μl of the recombinant plasmid DNA, and the mixture was loaded into a cuvette with a 4 mm electrode gap, and then was subjected to electric shock at 2.5 kV and 25 uF, followed by suspending immediately in 1 ml of 2×YTG medium to culture for 3 hours at 37° C.; thus, selecting transformants by spreading on a solid 2×YTG medium containing 40 μg/ml of erythromycin.

TABLE 2
Composition of CGM medium

| Component | Conc. (g/l) |
|---|---|
| Glucose | 80 |
| K₂HPO₄3H₂O | 0.982 |
| KH₂PO₄ | 0.75 |
| MgSO₄ | 0.348 |
| MnSO₄ H₂O | 0.01 |
| FeSO₄ 7H₂O | 0.01 |
| (NH₄)₂SO₄ | 2 |
| NaCl | 1 |
| Asparagines | 2 |
| PABA (paraaminobenzoic acid) | 0.004 |
| Yeast extract | 5 |

### Example 3

### Production of Ethanol/Butanol Using the Recombinant Microorganism M5(pIMP1::adhE1.ctfAB)

The recombinant microorganism M5(pIMP1::adhE1 ctfAB) prepared in Example 2 was cultured to examine the performance. A 30 ml test tube containing 10 ml of CGM medium was sterilized, taken out at a temperature higher than 80° C., charged with nitrogen gas, and cooled to room temperature in an anaerobic chamber. Then, 40 μg/ml of erythromycin was added to the medium, and the recombinant microorganism was inoculated, then preculture was carried out at 37° C. in an anaerobic condition to an absorbance of 1.0 at 600 nm. A 250 ml flask containing 100 ml of the medium with said composition was sterilized, the medium was inoculated with 6 ml of the preculture broth, and the second preculture was carried out at 37° C. in an anaerobic condition to an absorbance of 1.0 at 600 nm. Then, a 5.0 L fermentor (LiFlus GX, Biotron Inc., Kyunggi-Do, Korea) containing 2.0 L of the medium with said composition was sterilized, and cooled to room temperature while being supplied with nitrogen at 0.5 vvm, over a period of 10 hours, starting from a temperature higher than 80° C. after sterilization; then 40 μg/ml of erythromycin was added to the medium, followed by inoculating 100 ml of the second preculture broth to culture for 60 hours at 37° C. at 200 rpm. pH was maintained at 5.5 by automatic feeding of 5N NaOH, while nitrogen was supplied at 0.2 vvm (air volume/working volume/minute) throughout the culture.

The glucose in the medium was measured using a glucose analyzer (model2700 STAT, Yellow Springs Instrument, Yellow Springs, Ohio, USA); and an aliquot of the medium was taken out at various time points in order to measure the concentrations of acetone, ethanol and butanol produced therefrom, using a gas chromatography (Agillent 6890N GC System, Agilent Technologies Inc., CA, USA) equipped with a packed column (Supelco Carbopack™ B AW/6.6% PEG 20M, 2 m×2 mm ID, Bellefonte, Pa., USA).

As shown in the Table 3, the result showed that the control strain M5(pIMP1) did not produce ethanol and butanol, while the recombinant strain M5(pIMP1::adhE1.ctfAB) produced high concentrations of ethanol and butanol without producing almost no acetone (less than 0.5 g/l). Further, it was found that in addition to the high final concentrations of the produced ethanol and butanol, productivity was also improved.

Meanwhile, it is known that in the case of Clostridium acetobutylicum ATCC 824 strain, acetone production is about 28% of total organic solvent production (Harris et al., J. Ind. Microbiol. Biotechnol., 27:322, 2001); but in the case of the recombinant strain of the present invention, it was found that acetone production was less than about 5%, suggesting that the production thereof is negligible.

TABLE 3
Production of organic solvents by recombinant microorganisms

| | Strains and Production (g/l) | | |
|---|---|---|---|
| | M5 | M5 | |
| Solvent | (pIMP1) | (pIMP1::adhE1.ctfAB) | ATCC 824 |
| Acetone | 0.0 | 0.5 | 4.9 |
| Ethanol | 0.0 | 1.8 | — |
| Butanol | 0.0 | 8.0 | — |
| (ethanol + butanol)/ | 0% | 95% or more | 72% |
| (ethanol + butanol + | | | |
| acetone) | | | |

### INDUSTRIAL APPLICABILITY

As described above in detail, the present invention has the effect of providing a recombinant microorganism having the ability to produce ethanol and butanol with high yield through the introduction or amplification of specific genes. Based on manipulation of metabolic pathway, the recombinant microorganism according to the present invention shows not only almost no production of byproducts such as acetone, but also enhanced ethanol and butanol productivity per unit hour. Accordingly, the inventive microorganism is useful for industrial production of ethanol/butanol.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for constructing a recombinant microorganism having an enhanced ability to produce ethanol and butanol, the method comprises introducing a gene encoding an enzyme that converts acetic acid and butyric acid to acetyl CoA and butylyl CoA, respectively; and/or a gene encoding an enzyme that converts acetyl CoA and butyryl CoA to ethanol and butanol, respectively, into a host microorganism which has genes encoding enzymes involved in the biosynthetic pathway for conversion of acetyl CoA to butyryl CoA.

2. The method for constructing a recombinant microorganism having an enhanced ability to produce ethanol and butanol according to claim 1, wherein said biosynthetic pathway for conversion of acetyl CoA into butyryl CoA is [acetyl CoA→acetoacetyl CoA→3-hydroxybutyryl CoA→crotonyl CoA→butyryl CoA].

3. The method for constructing a recombinant microorganism having an enhanced ability to produce ethanol and butanol according to claim 1, wherein the host microorganism has an acetone biosynthetic pathway blocked.

4. The method for constructing a recombinant microorganism having an enhanced ability to produce ethanol and butanol according to claim 3, wherein the host microorganism has an adc (a gene encoding acetoacetic acid decarboxylase) deleted.

5. The method for constructing a recombinant microorganism having an enhanced ability to produce ethanol and butanol according to claim 1, wherein said host microorganism is derived from the genus Clostridium.

6. The method for constructing a recombinant microorganism having an enhanced ability to produce ethanol and butanol according to claim 1, wherein the enzyme converting acetic acid and butyric acid into acetyl CoA and butylyl CoA, respectively, is CoA transferase.

7. The method for constructing a recombinant microorganism having an enhanced ability to produce ethanol and butanol according to claim 6, wherein a gene encoding the CoA transferase is ctfAB.

8. The method for constructing a recombinant microorganism having an enhanced ability to produce ethanol and butanol according to claim 1, wherein the enzyme converting acetyl CoA and butyryl CoA into ethanol and butanol, respectively, is alcohol/aldehyde dehydrogenase.

9. The method for constructing a recombinant microorganism having an enhanced ability to produce ethanol and butanol according to claim 8, wherein a gene encoding the alcohol/aldehyde dehydrogenase is adhE1.

10. A recombinant microorganism having an enhanced ability to produce ethanol and butanol, which has a gene encoding an enzyme that converts acetic acid and butyric acid to acetyl CoA and butylyl CoA, respectively; and/or a gene encoding an enzyme that converts acetyl CoA and butyryl CoA to ethanol and butanol, respectively, introduced or amplified into a host microorganism having genes encoding enzymes involved in the biosynthetic pathway for conversion of acetyl CoA to butyryl CoA

11. The recombinant microorganism having an enhanced ability to produce ethanol and butanol according to claim 10, wherein said biosynthetic pathway for conversion of acetyl CoA into butyryl CoA is [acetyl CoA →acetoacetyl CoA→3-hydroxybutyryl CoA→crotonyl CoA→butyryl CoA].

12. The recombinant microorganism having an enhanced ability to produce ethanol and butanol according to claim 10, wherein the host microorganism has an acetone biosynthetic pathway blocked.

13. The recombinant microorganism having an enhanced ability to produce ethanol and butanol according to claim 12, wherein the host microorganism has an adc (a gene encoding acetoacetic acid decarboxylase) deleted.

14. The recombinant microorganism having an enhanced ability to produce ethanol and butanol according to claim 10, wherein said host microorganism is derived from the genus Clostridium.

15. The recombinant microorganism having an enhanced ability to produce ethanol and butanol according to claim 10, wherein the enzyme converting acetic acid and butyric acid into acetyl CoA and butylyl CoA, respectively, is CoA transferase.

16. The recombinant microorganism having an enhanced ability to produce ethanol and butanol according to claim 15, wherein a gene encoding the CoA transferase is ctfAB.

17. The recombinant microorganism having an enhanced ability to produce ethanol and butanol according to claim 10, wherein the enzyme converting acetyl CoA and butyryl CoA into ethanol and butanol, respectively, is alcohol/aldehyde dehydrogenase.

18. The recombinant microorganism having an enhanced ability to produce ethanol and butanol according to claim 17, wherein a gene encoding the alcohol/aldehyde dehydrogenase is adhE1.

19. The recombinant microorganism having an enhanced ability to produce ethanol and butanol according to claim 10, wherein its acetone production is less than 10% of the total organic solvent production

20. A recombinant Clostridium acetobutylicum M5(pIMP1::adhE1.ctfAB) which has an enhanced ability to produce ethanol and butanol

21. A method for preparing ethanol and/or butanol, the method comprising the steps of: culturing the recombinant microorganism of claim 10; and recovering ethanol and/or butanol from the culture broth.

22. A method for preparing ethanol and/or butanol, the method comprising the steps of: culturing the recombinant microorganism of claim 20; and recovering ethanol and/or butanol from the culture broth.
